(19) United States
(12) Patent Application Publication
(10) Pub. No.: US 2023/0329231 A1
(43) Pub. Date: Oct. 19, 2023

(54) A CUT FLOWER WITH PROLONGED VITALITY

(71) Applicant: OPTICEPT TECHNOLOGIES AB, Lund (SE)

(72) Inventors: Katarzyna Maria DYMEK-KRAKOWIAK, Lund (SE); Stephen KWAO, Lund (SE); Bengt EKELUND, Lund (SE); Ahmad HUSAIN, Lund (SE)

(21) Appl. No.: 18/025,464

(22) PCT Filed: Sep. 17, 2021
(86) PCT No.: PCT/SE2021/050892

§ 371 Date: Mar. 9, 2023

(30) Foreign Application Priority Data
Sep. 18, 2020 (SE) 2051094-7

(51) Int. Cl. A01N 3/02; A01P 1/00; A01P 21/00; A01N 59/16

(52) U.S. Cl. CPC A01N 3/02; A01P 1/00; A01P 21/00; A01N 59/16

(57) ABSTRACT

The present invention is directed to a cut flower with at least one leaf, wherein the silver content is at least 3.0 mg/kg dry substance in said at least one leaf, preferably at least 5.0 mg/kg dry substance in said at least one leaf, more preferably at least 7.0 mg/kg dry substance in said at least one leaf, more preferably at least 10.0 mg/kg dry substance in said at least one leaf, more preferably at least 15.0 mg/kg dry substance in said at least one leaf, more preferably at least 20.0 mg/kg dry substance in said at least one leaf, more preferably at least 25.0 mg/kg dry substance in said at least one leaf, most preferably at least 30.0 mg/kg dry substance in said at least one leaf.

### FIELD OF THE INVENTION

The present invention relates to a cut flower exhibiting prolonged shelf life after harvesting.

### TECHNICAL BACKGROUND

To prolong the shelf life of cut flowers after harvesting is of great interest. Today, it is a standard to obtain certain extra shelf life time for a bouquet of cut flowers by the addition of substances into the water containing holding the bouquet. The present invention is directed to providing cut flowers exhibiting prolonged vitality in comparison with what is obtainable today.

### SUMMARY OF THE INVENTION

The purpose above is achieved by the present invention. The present invention provides a cut flower with at least one leaf, wherein the silver content is at least 3.0 mg/kg dry substance in said at least one leaf, preferably at least 5.0 mg/kg dry substance in said at least one leaf, more preferably at least 7.0 mg/kg dry substance in said at least one leaf, more preferably at least 10.0 mg/kg dry substance in said at least one leaf, more preferably at least 15.0 mg/kg dry substance in said at least one leaf, more preferably at least 20.0 mg/kg dry substance in said at least one leaf, more preferably at least 25.0 mg/kg dry substance in said at least one leaf, most preferably at least 30.0 mg/kg dry substance in said at least one leaf.

It should be noted that the present invention is directed to cut flowers, implying flowers that have been harvested, and thus not plants or flowers in the ground, e.g. not flowers fixated in a flower-bed in a garden. Moreover, in relation to the method of the present invention, further disclosed below, this method is applied on one or more cut flowers, i.e. again on flowers which have been harvested.

According to the present invention, the silver content in the leaves is a strong marker for vitality and obtaining prolonged shelf-life. According to the present invention, cut flowers may be provided in which the leaves exhibit a silver content of at least 3.0 mg/kg dry substance in the leaves. This level is not obtained in cut flowers treated by procedures used today, and of course not naturally. As is notable from below, the present invention provides cut flowers in which one or more leaves has a silver content of at least 10.0 mg/kg dry substance, preferably at least 15.0 mg/kg dry substance, even as high as up to or above 20 mg/kg dry substance, and at certain stages during days after the treatment according to the present invention where the level is at least 25.0 mg/kg dry substance.

### SPECIFIC EMBODIMENTS OF THE INVENTION

Below there is provided some embodiments according to the present invention and also an explanation of the procedure, and embodiments thereof, how to obtain the cut flowers according to the present invention.

First of all, it should be mentioned that a cut flower treated according to the present invention will exhibit an increased silver content in at least the leaves after the treatment. This silver content may increase over the first days and will then eventually decrease again. The level as indicated above in the summary is set as a lowest possible level after treatment according to the present invention, although it is likely that the silver content according to the present invention will peak at a certain day after treatment and then decrease again after this. This also implies that a cut flower exhibiting a silver content as defined above, regardless if this is directly after the treatment or 5, or 10 or even 15 days after treatment, all of these cut flowers should be regarded as cut flowers covered by the definition presented above and thus treated according to the present invention.

To give a couple of concrete examples according to the present invention, these are presented in table 1 below.

TABLE 1

| | Silver content in leaves, |
|---|---|
| Sample | mg/kg dry matter |
| Cut flower sample 1 treated according | 37.32 |
| to the present invention, day 0 | |
| Cut flower sample 1 treated according | 51.71 |
| to the present invention, day 7 | |
| Cut flower sample 1 treated according | 23.10 |
| to the present invention, day 14 | |
| Cut flower sample 2 treated according | 32.49 |
| to the present invention, day 0 | |
| Cut flower sample 2 treated according | 20.07 |
| to the present invention, day 7 | |
| Cut flower sample 2 treated according | 40.50 |
| to the present invention, day 14 | |
| Naturally occurring in cut flower, | 2.79 |
| untreated control | |

For sample 1, the impregnation solution contained silver nitrate and a commercially available surfactant, namely Greenfain.

For sample 2, the impregnation solution contained silver nitrate and silica, and Greenfain.

It should be noted that different types of silver carrier substances and different types of surfactants may be used according to the present invention.

Although it should be noted that the exact measurement levels are not certain (see for instance the silver content level of sample 2 when comparing after 7 days and 14 days), which may be understood from the explanation of the measurement level below, it should be clear that the samples treated according to the present invention exhibit a silver content well above the naturally occurring level.

The measurement method was conducted as follows. The leaves, after treatment at certain days or without treatment in the control, were dried at 40° C. and then milled. Around 0.5 g of the milled material was digested with 7 ml concentrated Supra pure HNO₃ and 3 ml milli-Q water in closed vessels using a microwave, Mars 5 from CEM. The digested samples were then diluted up to 50 ml with water prior to analysis with ICP-OES, Optima 8300 from Perkin Elmer. It should be noted that Inductively Coupled Plasma Atomic Emission Spectroscopy (ICP-AES (also called ICP-OES)) is a technique to determine low-concentrations, and ultra-low-concentrations of elements in a mixture sample. Atomic elements are led through a plasma source where they become ionized. Then, these ions are sorted on account of their mass (there are already standards of mass for every known atom).

In line with the above, according to one embodiment of the present invention, there is disclosed a cut flower in which the silver content is at least 3.0 mg/kg dry substance in said at least one leaf, preferably at least 5.0 mg/kg dry substance in said at least one leaf, more preferably at least 7.0 mg/kg dry substance in said at least one leaf, more preferably at least 10.0 mg/kg dry substance in said at least one leaf, more preferably at least 15.0 mg/kg dry substance in said at least one leaf, preferably at least 20.0 mg/kg dry substance in said at least one leaf, as measured up to at least 7 days after treatment, preferably as measured up to 14 days after treatment.

Moreover, and as may be understood from above, according to the present invention the silver content in the leaves may be well above 10.0 mg/kg dry substance. In line with this, according to one specific embodiment of the present invention, the silver content is at least 10.0 mg/kg dry substance in said at least one leaf, preferably the silver content is at least 15.0 mg/kg dry substance in said at least one leaf, more preferably at least 25.0 mg/kg dry substance in said at least one leaf. Furthermore, according to yet another specific embodiment of the present invention, the silver content is at least 15.0 mg/kg dry substance in said at least one leaf, preferably at least 20.0 mg/kg dry substance in said at least one leaf, as measured up to at least 7 days after treatment, preferably as measured up to 14 days after treatment.

Moreover, according to the present invention also other parts of the cut flower may have an increased silver content. As an example, according to one specific embodiment of the present invention, the cut flower also comprises a stem and wherein the silver content is at least 3.0 mg/kg dry substance in the stem, preferably 10.0 mg/kg dry substance in the stem.

The cut flower product according to the present invention may be of different types. Of certain interest according to the present invention is a cut flower in the form of a rose, a tulip, an alstroemeria or a carnation, especially a rose.

As is hinted above, the present invention involves a specific treatment of the cut flower to enable to increase the silver content in at least the leaves of the cut flower. The method according to the present invention comprises
arranging one or more cut flowers in an impregnation solution which comprises at least one silver containing substance.

According to a preferred embodiment, the method comprises
arranging one or more cut flowers in an impregnation solution which comprises at least one silver containing substance (component); and
applying vacuum impregnation or pressure impregnation, preferably vacuum impregnation.

The method according to the present invention involves impregnation, preferably vacuum impregnation in an impregnation solution containing silver in one form or another. Moreover, according to one preferred embodiment, the impregnation solution also comprises at least one surfactant. By using a surfactant according to the present invention, the surface tension of the impregnation solution is broken, which in turn enables to provide much more of the active components (treatment liquid) into the leaves and stem of the cut flower. It is this starting point according to the present invention which enables to increase the active silver component in the leaves.

Furthermore, when performing the vacuum impregnation, gases, such as air, inside of the cut flower is replaced with liquid. The liquid or impregnation solution may have different functions depending in the components contain in the impregnation solution. Possible functionalities are to add nutrition and prevent growth of bacteria and fungus. Furthermore, it may work as an ethylene blocker. In this regard it should be noted that silver has the ability to block ethylene responses in plants, proving the great effect of the concept according to the present invention.

By treating the leaves according to the present invention an osmosis reaction occurs. This in turn implies that the cut flower increases water intake, which in turns implies a longer shelf life.

The vacuum impregnation method according to the present invention is performed in a treatment chamber. The pressure is then decreased to a certain minimum pressure, where air leaves different voids in the cut flower being treated. At the same time, the treatment chamber is filled with impregnation solution. The minimum pressure is held for a certain short period of time and when the pressure then is increased again the voids are filled with the impregnation solution instead of air. Again, the use of a surfactant according to the present invention implies that more active component, i.e. at least silver, penetrates into the cut flower, at least into the leaves and stem(s) of the cut flower.

Moreover, silver may be provided into the impregnation solution in different forms. One example is as silver nitrate being provided into the impregnation solution.

Furthermore, different types of surfactants may be used. According to one embodiment of the present invention, said at least one surfactant is an anionic surfactant. Anionic surfactants which are readily biodegradable may be relevant to use according to the present invention.

Moreover, it may also be of interest how to perform the actual impregnation. According to one specific embodiment of the present invention, the step of arranging one or more cut flowers in an impregnation solution is performed so that at least a portion of the stem of said one or more cut flowers are immersed in the impregnation solution, but where the flower bud is free from impregnation solution, and wherein the step of applying vacuum impregnation or pressure impregnation, preferably vacuum impregnation, is performed to the impregnation solution when said at least portion of the stem are immersed into the impregnation solution. According to this embodiment, the method ensures that the flower bud is free from impregnation solution. This may also be of importance in some case and is a unique aspect of the present invention.

In addition to said at least one surfactant, the impregnation solution may also comprise other substances. First of all, the impregnation is suitably an aqueous liquid comprising at least one sugar, preferably wherein said at least on sugar is glucose, trehalose and/or fructose, or a sugar alcohol, preferably sorbitol, or a combination thereof.

Moreover, the impregnation solution may also comprise additives. In relation to the impregnation solution it should be noted that this may comprise at least one sugar without any additives, at least one sugar and additives, or only additives. At least one surfactant is, however, always present to obtain the enhanced results of silver content in the leaves.

In relation to additives, many different are possible. According to one specific embodiment of the present invention, the impregnation solution comprises at least one additive being a vitamin, mineral, ethylene controller, antioxidant, hormone, e.g. a growth hormone, nutrient, antimicrobial, fertilizer, or a combination thereof.

Some additional specific alternatives of interest are presented below.

### Growth Regulators

Paclobutrazol (PBZ)

Naphthaleneacetic acid (NAA)

Indole butyric acid (IAA)

Thidiazuron (TDZ)

Benzylaminopurine (BAP)

Adenine

Gibberellins (GA:s)

### Stress Response Regulators

Jasmonic acid (JA)

Salicylic acid (SA)

All of the above are possible in different combinations according to the present invention.

According to yet another specific embodiment of the present invention, the impregnation solution comprises at least one additive of folic acid, gamma-aminobutyric acid (GABA), 1-methylcyclopropene (1-MCP), or a combination thereof.

Furthermore, method process parameters are also of interest according to the present invention. According to one embodiment, the method involves vacuum impregnation in a minimum pressure range of 50-500 mbar, preferably in the range of 60-300 mbar. According to yet another embodiment, the method is performed during a treatment time of at least 5 seconds, preferably in the range of 5 seconds—15 minutes, more preferably in the range of 5 seconds—5 minutes. Furthermore, according to yet another specific embodiment, the method involves vacuum impregnation in at least three phases, said at least three phases being a falling step when the pressure is decreased to a certain low pressure, then a holding step in which the low pressure is kept or substantially kept at the low pressure, and a pressure rising step where the pressure is increased to atmospheric level.

Other steps may also be part of the method according to the present invention. According to one specific embodiment, the method involves a subsequent washing step comprising immersing said one or more cut flowers into water to wash sugars and/or other substances from the surface of the cut flower, and wherein the immersing is performed so that the flower bud is excluded from being immersed. Moreover, according to another embodiment, the cut flowers are directly subjected to a cooling step after the washing step, said cooling step being a recovering step. The cooling step may suitably be performed at a temperature of 2-10° C., such as suitably in a range of 5-10° C. Moreover, according to one embodiment, the cooling step is performed during at least 6 hours, preferably at least 12 hours, such as in the range of 12-24 hours. Furthermore, the cooling should be performed in a controlled storing environment. According to one embodiment, the storing environment involves a humidity of above 50%. Moreover, the cooling and transportation may be performed in a modified atmosphere. Furthermore, the cooling may suitably be performed in an aerated room so that the surfaces of the leaves dry on themselves.

In relation to the above it should be mentioned that the cooling step may also be seen as a recovery step in a cooled or low temperature.

Moreover, according to yet another embodiment, freezing is applied instead of cooling. Cooling is, however, preferred.

Moreover, according to yet another embodiment, the impregnation is a partial impregnation, preferably wherein the impregnation is a partial impregnation where the weight gain is 50% of the full impregnation weight gain.

Prevention of microbial contamination is important. According to one embodiment of the present invention, the method also comprises an active step for preventing microbial contamination of the (aqueous) impregnation solution. According to one specific embodiment, the active step for preventing microbial contamination involves adding one or more antimicrobial agents to the impregnation solution, preferably wherein the active step for preventing microbial contamination involves an active treatment of the impregnation solution. Moreover, according to yet another embodiment, the impregnation solution is recirculated and reused, preferably as an active step for preventing microbial contamination.

According to one specific embodiment of the present invention, the method is free from performing a PEF (pulsed electric field) step. PEF may be involved after the impregnation step, but this is optional. In this regard it should be noted that only the impregnation is mandatory. Therefore, according to yet another specific embodiment, the method also involves a step of applying PEF (pulsed electric field).

1. A cut flower with at least one leaf, wherein the silver content is at least 3.0 mg/kg dry substance in said at least one leaf, preferably at least 5.0 mg/kg dry substance in said at least one leaf, more preferably at least 7.0 mg/kg dry substance in said at least one leaf, more preferably at least 10.0 mg/kg dry substance in said at least one leaf, more preferably at least 15.0 mg/kg dry substance in said at least one leaf, more preferably at least 20.0 mg/kg dry substance in said at least one leaf, more preferably at least 25.0 mg/kg dry substance in said at least one leaf, most preferably at least 30.0 mg/kg dry substance in said at least one leaf.

2. The cut flower according to claim 1, wherein the silver content is at least 10.0 mg/kg dry substance in said at least one leaf, preferably the silver content is at least 15.0 mg/kg dry substance in said at least one leaf, more preferably at least 25.0 mg/kg dry substance in said at least one leaf.

3. The cut flower according to claim 1 or 2, wherein the silver content is at least 3.0 mg/kg dry substance in said at least one leaf, preferably at least 5.0 mg/kg dry substance in said at least one leaf, more preferably at least 7.0 mg/kg dry substance in said at least one leaf, more preferably at least 10.0 mg/kg dry substance in said at least one leaf, more preferably at least 15.0 mg/kg dry substance in said at least one leaf, preferably at least 20.0 mg/kg dry substance in said at least one leaf, as measured up to at least 7 days after treatment, preferably as measured up to 14 days after treatment.

4. The cut flower according to claim 1, wherein the silver content is at least 15.0 mg/kg dry substance in said at least one leaf, preferably at least 20.0 mg/kg dry substance in said at least one leaf, as measured up to at least 7 days after treatment, preferably as measured up to 14 days after treatment.

5. The cut flower according to claim 1, wherein the cut flower also comprises a stem and wherein the silver content is at least 10.0 mg/kg dry substance in the stem.

6. The method for the production of a cut flower according to claim 1, said method comprising
arranging one or more cut flowers in an impregnation solution which comprises at least one silver containing substance.

7. The method for the production of a cut flower according to claim 6, said method comprising
arranging one or more cut flowers in an impregnation solution which comprises at least one silver containing substance; and
applying vacuum impregnation or pressure impregnation, preferably vacuum impregnation.

8. The method according to claim 6, wherein the impregnation solution comprises at least one surfactant.

9. The method according to claim 8, wherein said at least one surfactant is an anionic surfactant.

10. The method according to claim 6, wherein the step of arranging one or more cut flowers in an impregnation solution is performed so that at least a portion of the stem of said one or more cut flowers are immersed in the impregnation solution, but where the flower bud is free from impregnation solution, and wherein the step of applying vacuum impregnation or pressure impregnation, preferably vacuum impregnation, is performed to the impregnation solution when said at least portion of the stem are immersed into the impregnation solution.

11. The method according to claim 6, wherein the impregnation solution comprises at least one sugar, preferably wherein said at least on sugar is glucose, trehalose and/or fructose, or a sugar alcohol, preferably sorbitol, or a combination thereof.

12. The method according to claim 6, wherein the impregnation solution comprises at least one additive being a vitamin, mineral, ethylene controller, antioxidant, hormone, e.g. a growth hormone, nutrient, antimicrobial, fertilizer, or a combination thereof.

13. The method according to claim 6, wherein the impregnation solution comprises at least one additive of folic acid, gamma-aminobutyric acid (GABA), 1-methylcyclopropene (1-MCP), or a combination thereof.

14. The method according to claim 6, wherein the method involves vacuum impregnation in a minimum pressure range of 60-300 mbar.

15. The method according to claim 14, wherein the method is performed during a treatment time of at least 5 seconds, preferably in the range of 5 seconds—15 minutes, more preferably in the range of 5 seconds—5 minutes.

16. The method according to claim 6, wherein the method is free from performing a PEF (pulsed electric field) step.

17. The method according to claim 6, wherein the method also involves a step of applying PEF (pulsed electric field).

18. The method according to claim 6, wherein the method involves a subsequent washing step comprising immersing said one or more cut flowers into water to wash sugars and/or other substances from the surface of the cut flower, and wherein the immersing is performed so that the flower bud is excluded from being immersed.

19. The method according to claim 18, wherein the cut flowers are directly subjected to a cooling step after the washing step, said cooling step being a recovering step.

20. The method according to claim 19, wherein the cooling step is performed at a temperature of 5-10° C., preferably wherein the cooling step is performed during at least 6 hours, preferably at least 12 hours.
